(19) United States
(12) Patent Application Publication
(10) Pub. No.: US 2023/0288085 A1
(43) Pub. Date: Sep. 14, 2023

(54) AIR SAFETY SYSTEMS

(71) Applicant: ITT Manufacturing Enterprises LLC, Wilmington, DE (US)

(72) Inventors: A. David SANDIFORD, Valencia, CA (US); Hsuannien Samuel CHEN, Valencia, CA (US); Steve E. KIM, Valencia, CA (US)

(73) Assignee: ITT Manufacturing Enterprises LLC, Wilmington, DE (US)

(21) Appl. No.: 18/017,976

(22) PCT Filed: Nov. 18, 2020
(86) PCT No.: PCT/US20/60942

§ 371 Date: Jan. 25, 2023

Related U.S. Application Data
(60) Provisional application No. 63/067,399, filed on Aug. 19, 2020.

(51) Int. Cl. F24F 9/00; A61G 10/02; A61L 9/20; F24F 8/22

(52) U.S. Cl. CPC F24F 9/00; A61G 10/02; A61L 9/20; F24F 8/22; A61L 2209/14

(57) ABSTRACT

Technologies are described for providing safe and clean air and to prevent interchange of fluids or particulates between people in social situations through two or more distinct airflows generated and/or directed by various structures. An air safety system may receive pre-sanitized air and push through a guidance structure to create a low-velocity, clean air safe zone that prevents exchange of fluids and particulates within the zone and a high speed air shield to prevent injection of fluids and particulates from outside into the safe zone. The system may accommodate one or more persons in an open space of over a table/desk. A modular air safety system may generate a similar safe zone and air shield horizontally in a smaller scale for one or more people.

### CROSS-REFERENCE TO RELATED APPLICATIONS

This application claims priority to U.S. Provisional Patent Application Ser. No. 63/067,399 filed on Aug. 19, 2020. The disclosure of the Provisional Application is hereby incorporated by reference in their entirety.

### BACKGROUND

Unless otherwise indicated herein, the materials described in this section are not prior art to the claims in this application and are not admitted as prior art by inclusion in this section.

COVID-19 pandemic is an extreme example of how an infectious disease, specifically, and airborne infection can disrupt daily life and activities. Masks and face shields provide some protection, but have limitations and disadvantages associated with them. For example, when eating, a mask or face shield has to be removed. Thus, restaurants and similar eating establishments (and their customers) either have to take a risk stop operating. People who rely on lip reading (deaf or hard-of-hearing) cannot understand others in social environments. Younger students find it hard to wear a mask during an entire school day. There are many examples of hardships caused by wearing masks or face shields. On the other hand, in case of a serious disease like COVID-19, the alternative may be serious complications or even death.

### SUMMARY

The present disclosure generally describes various structures to provide safe and clean air and to prevent interchange of fluids or particulates between people in social situations through two or more distinct airflows generated and/or directed by the various structures.

According to some examples, an air safety system may include an air source configured to receive air and direct the air to the air safety system; a pressurization module configured to receive the air from the air source and direct the air at a predetermined pressure to a guidance structure; and the guidance structure comprising two or more channels. The guidance structure, through the two or more channels, may be configured to generate a first airflow in a central column region with a first pressure to create a safe zone with low velocity, sanitized air for one or more users within the central column region; and generate a second airflow to form an air shield around the central column region with a second pressure higher than the first pressure to prevent entry of fluids and particulates from an external environment into the safe zone. The air safety system may include an air source configured to receive pre-sanitized air and direct the pre-sanitized air to the air safety system. Alternatively, the air source may be an air intake module configured to receive air from the external environment, where the air safety system may further include a sanitization module comprising one or more of a filter module to filter the received air and an ultra-violet (UV) module to apply UV light to the received air. The sanitization module may sanitize the received air as the air passes from the air source to the pressurization module.

According to other examples, a modular air safety system may include a support module with an air intake opening configured to receive air; a pressurization module to direct the air at a predetermined positive pressure; and one or more airflow nozzles coupled to the support module through a joint. The one or more airflow nozzles may include a central flow portion having a shaped wall that defines an intake opening and couples to an emitter opening defined by the shaped wall, where the coupling is angled and has an increasing flow area; and a boundary flow portion surrounding the central flow portion and having another shaped wall that defines another intake opening and couples to another emitter opening defined by the other shaped wall, where the coupling is angled and has a decreasing flow area. The central flow portion may be configured to reduce a speed of received airflow and provide sanitized air toward a region of interest, and the boundary flow portion may be configured to increase a speed of received airflow and provide an air shield around the region of interest to prevent entry of fluids and particulates from an external environment.

According to further examples, a method to create a safe zone with sanitized air may include receiving pre-sanitized air from a pre-sanitized air source; pressurizing the received pre-sanitized air at a predetermined pressure to a guidance structure; generating a first airflow in a central column region with a first pressure to create a safe zone with low velocity, sanitized air for one or more users within the central column region by guiding the pressurized air through one or more channels of the guidance structure; and generating a second airflow to form an air shield around the central column region with a second pressure higher than the first pressure to prevent entry of fluids and particulates from an external environment into the safe zone by guiding the pressurized air through one or more other channels of the guidance structure.

According to yet other examples, a method to create a safe zone with sanitized air may include receiving air from an external environment; sanitizing the received air through a sanitization module comprising one or more of a filter module to filter the received air and an ultra-violet (UV) module to apply UV light to the received air; pressurizing the sanitized air at a predetermined pressure to a guidance structure; generating a first airflow in a central column region with a first pressure to create a safe zone with low velocity, sanitized air for one or more users within the central column region by guiding the pressurized air through one or more channels of the guidance structure; and generating a second airflow to form an air shield around the central column region with a second pressure higher than the first pressure to prevent entry of fluids and particulates from an external environment into the safe zone by guiding the pressurized air through one or more other channels of the guidance structure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIGS. 1A and 1B illustrate conceptual cross-sectional views of example single-user and multi-user air safety systems in vertical and horizontal configurations;

FIG. 2 illustrates a cross-sectional view of an example air safety system;

FIG. 3 illustrates airflows in two configurations of the example air safety system of FIG. 2;

FIG. 4A through 4D illustrate various views, airflows, and fluid dynamic flow simulations for an example multi-user air safety system in a table configuration;

FIGS. 5A and 5B illustrate various views and fluid dynamic flow simulations for an example modular air safety system;

FIGS. 6A and 6B illustrate a perspective view and fluid dynamic flow simulations for another example modular air safety system; and

FIG. 7 illustrates an array configuration of the example modular air safety system of FIG. 5A, arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems and/or devices related to providing safe and clean air and to prevent interchange of fluids or particulates between people in social situations through two or more distinct airflows generated and/or directed by various structures.

The COVID-19 pandemic has negatively affected many business and personal interactions. For example, the traditional restaurant business has been decimated. Wearing a face mask impedes even simple interactions with a cashier or across a desk in a business setting. A large part of human communication involves facial visual recognition as well as clear audio of the spoken word. As this airborne virus and similar infectious diseases are spread through interchange of air and particles between people via a combination of close proximity, speaking, coughing and breathing among a group of people gathered together, for example, at a communal table for eating, business meetings, interviews, and similar ones. Another challenge is communication. Speaking to a small group of “socially distanced” people from behind a desk almost requires shouting to get past the acoustic attenuation of a face covering.

At the individual user level, embodiments are directed to delivery of sterilized, low velocity positive pressure air in a limited zone to the user for breathing. Through manipulation of the boundary conditions, either by the aerodynamic shape of an enclosure or an air outlet shape to accelerate and guide a boundary zone condition, introduction of ambient and possibly contaminated air to the safe breathing zone may be prevented.

In some examples, an air safety system may include an air source configured to receive pre-sanitized air and direct the pre-sanitized air at a predetermined positive pressure to a containment structure. The containment structure may include a hollow chamber, an air intake opening coupled to the air source and two or more user openings to direct portions of the pre-sanitized air toward two or more users positioned external to the two or more user openings. The containment structure may guide the pre-sanitized air downward from a top portion such that an airflow is created in a central column region within the chamber with a pressure sufficient to prevent interchange of fluids and particulates between two or more users in the safe zone. The containment structure may also guide the pre-sanitized air downward along interior walls of the chamber such that two or more other airflows are created to exit through the two or more openings at a higher pressure sufficient to generate a sanitized air shield around an upper body of each of the two or more users.

In other examples, instead of taking in pre-sanitized air, the air safety system may receive un-sanitized air from the external environment and include a sanitization module. The sanitization module may include a filter module to filter the received air and an ultra-violet module to apply UV light to the received air. The pressurization module may direct the sanitized air at a predetermined pressure to the containment structure.

In further examples, a modular air safety system may include a support stand that includes an air intake opening configured to receive air from a pre-sanitized air source and a pressurization module to direct the pre-sanitized air at a predetermined positive pressure out of the support stand. The modular air safety system may also include an airflow nozzle coupled to the support stand through a joint. The airflow nozzle may include a central flow portion having a shaped wall that defines an intake opening and couples to an emitter opening defined by the shaped wall. The coupling may be angled and have an increasing flow area. The airflow nozzle may also have a boundary flow portion surrounding the central flow portion and having another shaped wall that defines another intake opening and couples to another emitter opening defined by the other shaped wall. The coupling may also be angled and have a decreasing flow area. The central flow portion may be configured to reduce a speed of received airflow and provide sanitized air toward a region of interest, and the boundary flow portion may be configured to increase a speed of received airflow and provide a conically shaped sanitized air shield around the region of interest from external un-sanitized air.

In yet other examples, instead of taking in pre-sanitized air, the modular air safety system may receive un-sanitized air from the external environment and include a sanitization module. The sanitization module may include a filter module to filter the received air and an ultra-violet module to apply UV light to the received air. The modular air safety system may also include a pressurization module to direct the sanitized air at a predetermined pressure to the airflow nozzle.

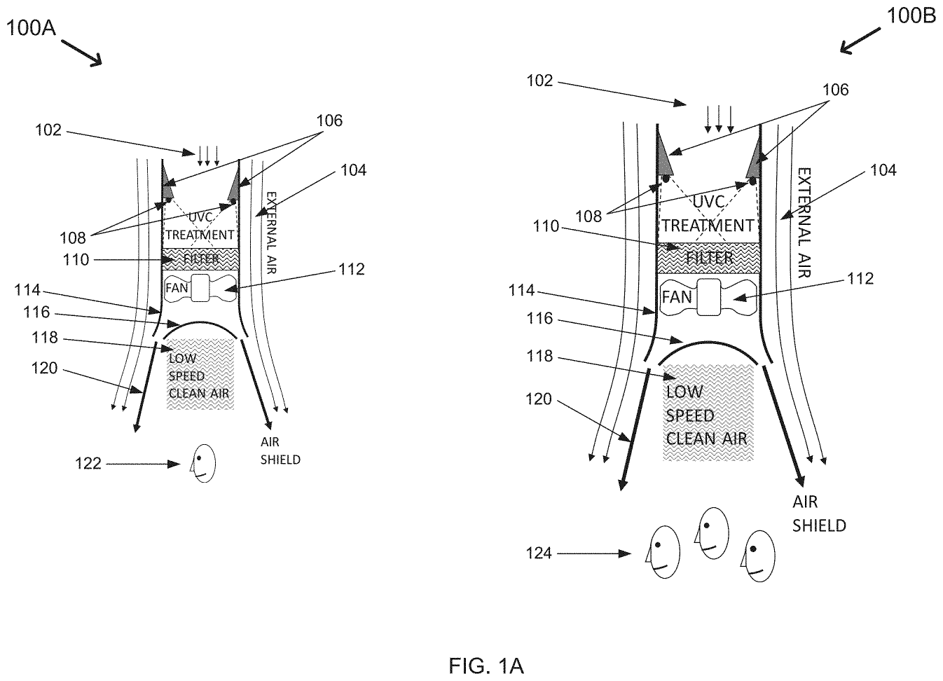

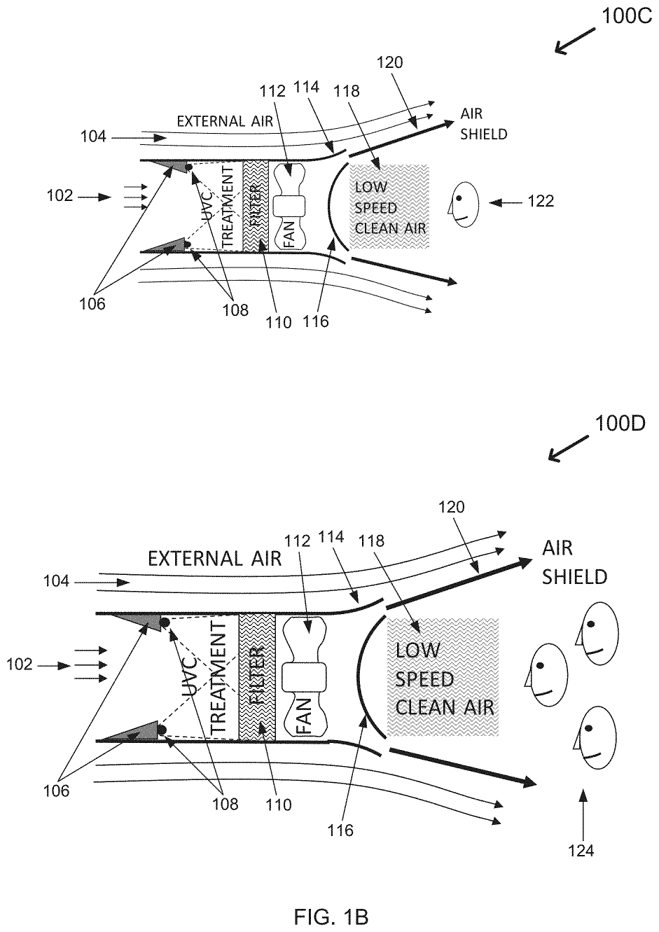

FIGS. 1A and 1B illustrate conceptual cross-sectional views of example single-user and multi-user air safety systems in vertical and horizontal configurations, arranged in accordance with at least some embodiments described herein.

An example system may employ High Volume, Low Pressure (HVLP) airflow technology along with the aerodynamic wall design that contains the fluid flow and allows close interaction and other activities without face coverings. Sanitization may be accomplished through filtering (e.g., HEPA filter) and UV-C radiation. To allow for easy communication, noise levels in the pressurization module may be controlled (e.g., <50 dB). The system may be scaled, modularized or shape changed for different implementation environments and usage types. Furthermore, the open design may allow for user personalization.

To supply separated, individual purified air to a varied number of users, with differing heights, without simply zoning off each user into a physically bounded individual box, the system may maintain a physical method of preventing the interaction or transfer of virus particle, mucous droplets, a sneeze or other fluid transfer between users. High velocities in traditional air curtain or air knife systems typically generate very high background noise levels disturbing vocal communication while in the protected area along with other negative impacts of high velocity air on any items (e.g. papers, food, etc.) in the area. The low velocity of a system according to embodiments overcomes such challenges.

As shown in diagrams 100A, 100B, 100C, and 100D, a single-user or a multi-user air safety system according to embodiments may provide sanitized air to a single person or a group of people sitting or standing within a pressure differential zone. The system creates a localized environment for individuals or small groups of people in places such as restaurants, offices, other workplaces, government offices, hospitals, dental or similar medical offices, classrooms, buses, airplanes, ships, trains, casinos, etc. with clean, safe air separated from surrounding potentially contaminated air.

Diagram 100A shows an example single-user configuration generating a low speed, clean (sanitized) air safe zone vertically for an individual. Thus, the system may be installed at a ceiling or similar height and provide the safe zone for the individual below the system. In the shown configuration, un-sanitized air 102 may be received from the external environment into an air intake module 106 comprising a sanitization module that includes UV-C treatment through UV lamps or diodes (108) and a filter 110 (e.g., a HEPA filter). The sanitized air may be pushed down at a predetermined pressure by a pressurization module, which may include a fan 112 or other mechanisms such as air multipliers. A guidance structure formed by shaped walls 114 and 116 may generate two different pressure zones. A first pressure zone with lower speed may create the safe zone 118 and a second higher pressure zone 120 may create the air shield around the safe zone 118 and person 122. The air shield 120 may prevent un-sanitized external air 104 from entering the safe zone along with any fluids or particulates that may be coming from other people in the area (e.g., through their breathing, sneezing, etc.).

Diagram 100B shows a scaled-up version of the single-person configuration of diagram 100A for multiple people 124. A size, shape, or various dimensions of the guidance structure may be modified to scale up the system along with sizes of the components of the sanitization and pressurization modules. Furthermore, a more powerful fan (or air multipliers) or multiple fans may also be used to accommodate increased airflow for the larger volume. Similarly, number or strength of UV-C sources may also be adjusted.

Examples of multiple-user systems may include systems for a family or a similar group of people, who do not suspect each other being infected, may be comfortable in being within the same safe zone even though they may exchange fluids or particulates. The group may still be protected from fluids or particulates in the external environment.

Diagram 100C shows the configuration of Diagram 100A in a horizontal configuration with components, arrangements, and functionalities similar between the two. The horizontal configuration may be installed at a workstation (e.g., on a desk or workbench), in a vehicle, at a public use location (e.g., at a cash machine or similar public terminal), and comparable locations.

Diagram 100D is a scaled-up version of the single-person configuration of diagram 100C for multiple people 124. A size, shape, or various dimensions of the guidance structure along with sizes and strengths of various components of the components of the sanitization and pressurization modules may be modified to scale up the system. Example implementations for the configuration in diagram 100D may include, but are not limited to, workstations, vehicles (e.g., airplane seat rows), and similar ones.

In some embodiments, a shape and size of the safe zone 118 and/or the air shield 120 may be selected or adjusted by selecting shape and size of the shaped walls 114 and 116 of the guidance structure. Number of fans or power of the fan may also be selected for suitable safe zone and air shield sizes. An example system may be implemented at other angles beside vertical or horizontal as well. A size and shape of the safe zone may be selected (or adjusted) according to the number of people to be covered, an expected distance of the group from the system, etc.

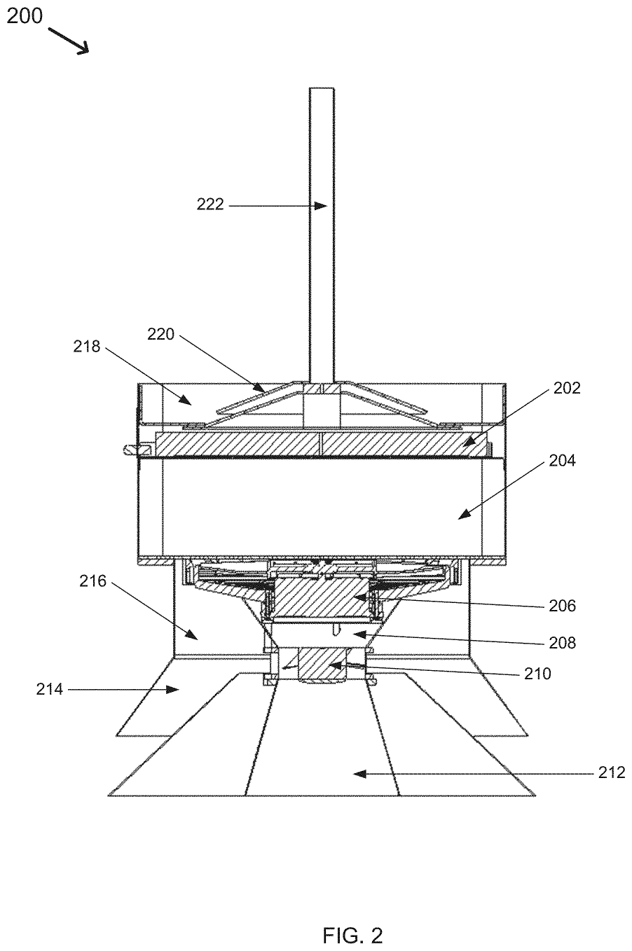

FIG. 2 illustrates a cross-sectional view of an example air safety system, arranged in accordance with at least some embodiments described herein.

Diagram 200 shows a practical configuration for a ceiling-hung air safety system with dual fans. The system may be hung from the ceiling through down pipe 222, which may also be used to pass through power and control cables. A first stage (upper plenum 218) of the system may house a UV-C reflector assembly 220. A filter 202 (e.g., HEPA filter) may be positioned in a slidable drawer 204 for servicing or replacement. Lower plenum 216 may include primary fan 206 and secondary fan inlet collector 208. The smaller secondary fan 210 may push sanitized air into the secondary fan emitter cone 212 while the primary fan 206 pushes sanitized air into the primary fan emitter cone 214.

Atmospheric pressure air from the environment is drawn into the air safety system through the inlet area in the upper plenum 218 of the system. The UV-C light disinfects the air along with the filter 202 and the inlet area. The primary fan 206 pulls the sanitized air through the filter 202 and pressurizes it for both the central flow area and the boundary flow generator area. The airflow in the boundary flow generator is low velocity until it enters the compression area in the primary fan emitter cone 214, where it is compressed by space reduction and then exits the primary fan emitter cone 214 around the periphery of the emitter. The central flow area starts out with high speed airflow as it enters the secondary fan emitter cone 212, which is then reduced by expansion. The high volume, low velocity air for the central bubble is emitted through the secondary fan emitter cone 212.

As shown in diagram 200, forming the central/peripheral flow fields can also be accomplished through the use of multiple fans, either sharing the filter/sanitation module or separately. The primary fan 206 forms the high-speed peripheral flow and the secondary inner fan 210 is adjusted for proper fill and to prevent inward collapse of the peripheral flow due to an insufficient pressure gradient.

The cones may be vacuum formed plastic, for example, acrylic or similar materials. The filter box (drawer) may be a plastic vacuum form, seam-welded aluminum, or comparable construction. The filter supports, drawer, lower fan support and UV-C LED reflector may be made from aluminum. In some examples, power supply circuits and control electronics may be placed in the section above the UV-C reflector 220. In other examples, the high-pressure fan may be combined with both the inlet and outlet cones and the entire assembly may be fastened through the high-velocity outer fan cover. The air safety system may also be combined with lighting and/or sound systems to provide lighting and/or sound (e.g., music) to the area below the system. For example, various lighting color schemes may be employed for different environments (e.g., restaurants, bars, workplaces, schools, etc.)

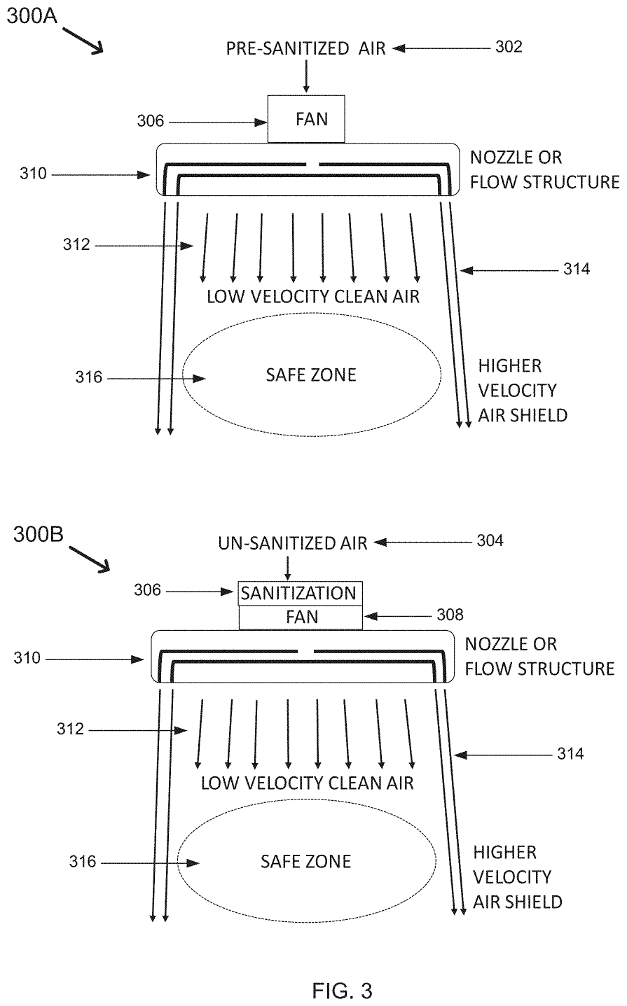

FIG. 3 illustrates airflows in two configurations of the example air safety system of FIG. 2, arranged in accordance with at least some embodiments described herein.

In example air safety systems, sanitized air may be supplied to the system under high volume, with low velocity. The users are positioned in the positive pressure area and are continuously enveloped in a column of sanitized air. The low velocity, clean air safe zone 316 allows users to work, converse, eat, and perform other actions with a risk of exchange of fluids and particulates between users being minimized. The higher velocity air shield 314 surrounding the safe zone 316 protects the users from fluids and particulates that may be in the external environment.

In FIG. 3, airflows of two example configurations are shown. First configuration in diagram 300A takes in pre-sanitized air 302 and pushes the pre-sanitized air through a pressurization system (fan 306) into the guidance structure 310 creating the safe zone 316 with low velocity clean air 312 and the higher velocity air shield 314 in the periphery. The second configuration in diagram 300B takes in un-sanitized air 304 from the external environment, sanitizes in a sanitization module 308 (e.g., UV-C treatment and HEPA filtering), and then pushes the sanitized air through a pressurization system (fan 306) into the guidance structure 310 creating the safe zone 316 with low velocity clean air 312 and the higher velocity air shield 314 in the periphery. The guidance structure may include multiple channels to guide the pressurized air and affect it pressure/speed through widening or narrowing walls.

In some embodiments, the alignment of the airflow may be directed in any desired direction in the XYZ axis. Incoming air may pass through a number of activated charcoal or HEPA filters and be guided through a labyrinthine path to prevent the UV-C light from escaping to the outside as UV-C exposure is damaging to the eyes and skin. As filters may need to be regularly maintained/replaced to ensure proper operation, they may be easily accessible (e.g., through a drawer structure as described above). An interlock may prevent the UV-C sources from operating when the plenum or any upper area is being serviced.

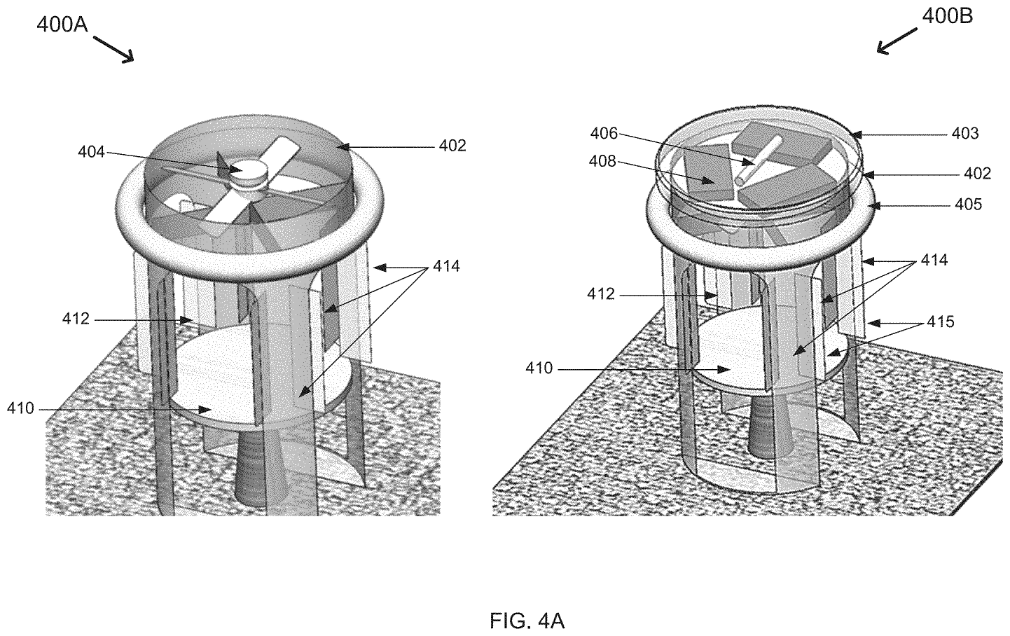

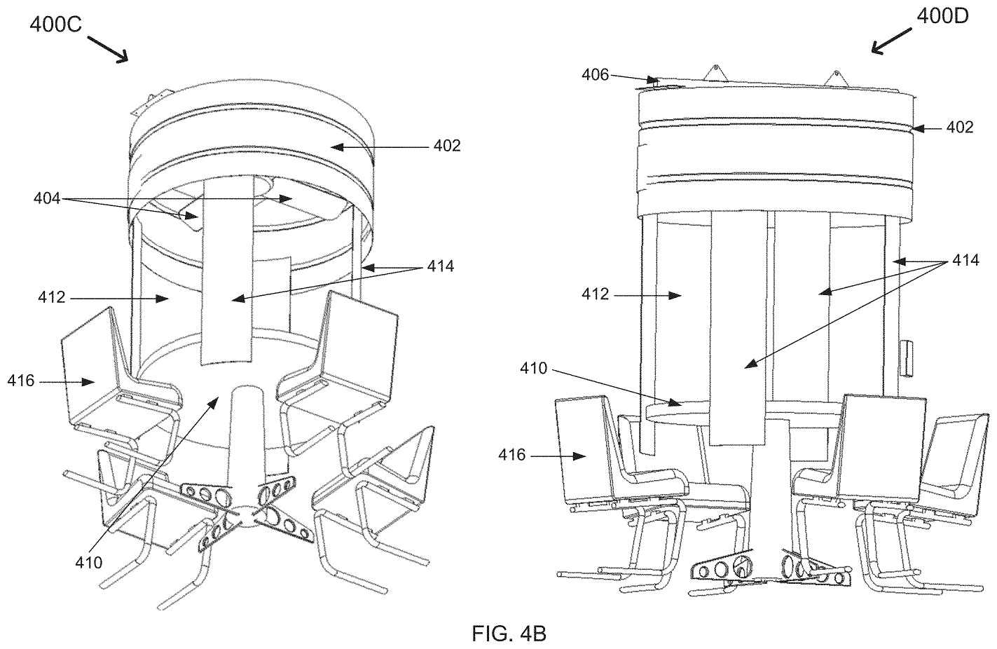

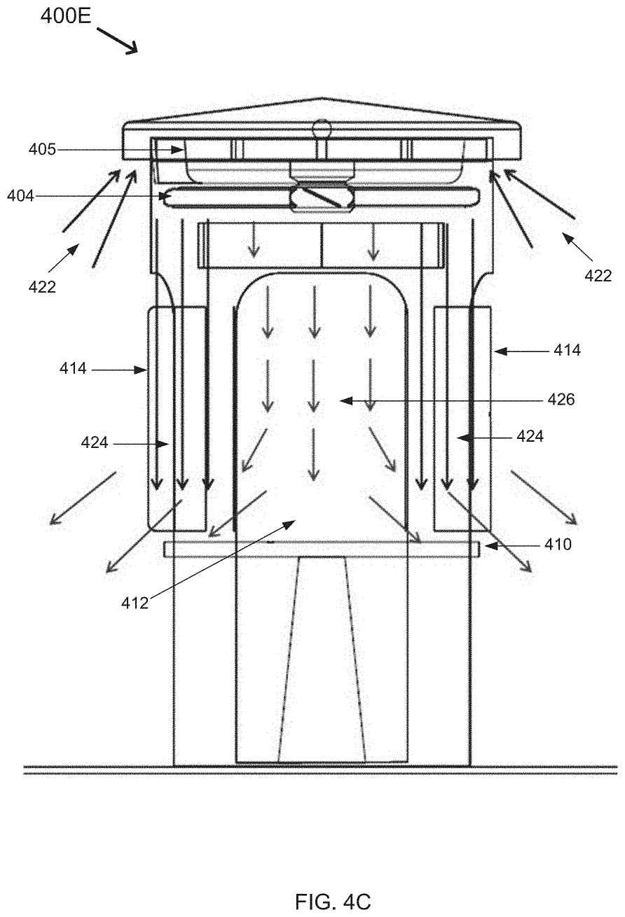

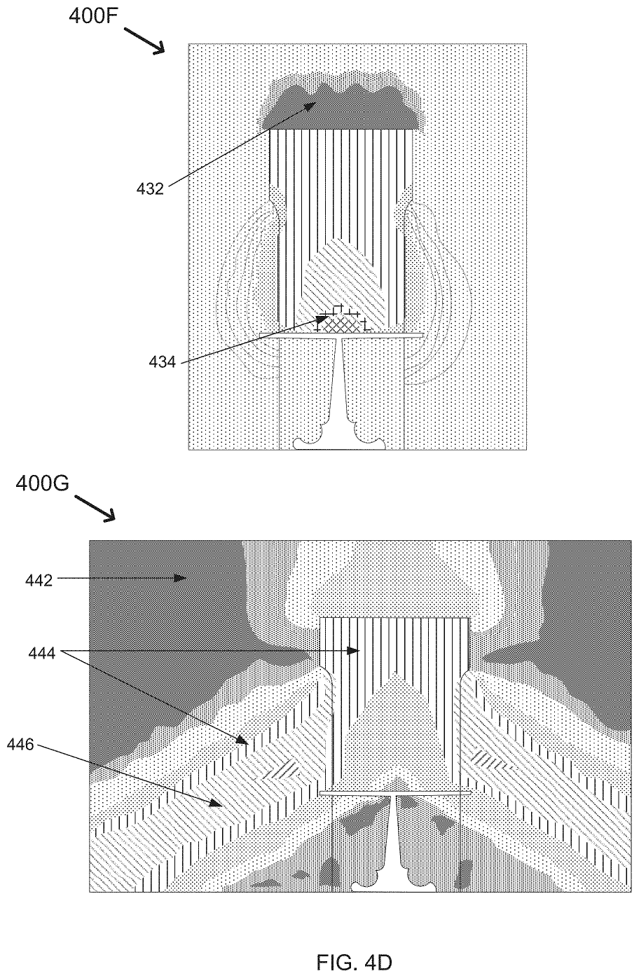

FIG. 4A through 4D illustrate various views, airflows, and fluid dynamic flow simulations for an example multi-user air safety system in a table configuration, arranged in accordance with at least some embodiments described herein.

Diagram 400A in FIG. 4A illustrates side elevation views of an example multi-user air safety system in a table configuration with various components. Pressurization module 402 may include one or more fans 404 to push the air down into a containment enclosure defined by walls 414 and user openings 412. In the table configuration, the containment enclosure may be positioned on a table 410 allowing users to stand or sit at the table 410 with a low-velocity, clean air safe zone being created over the table and an air shield around the table. In the configuration of diagram 400A, a sanitization module is not shown, thus the pressurization module 402 may be configured to receive pre-sanitized air from a pre-sanitization air source. For example, a single sanitization module may be coupled to multiple systems (tables) at a location.

Diagram 400B in FIG. 4A illustrates the same configuration with a sanitization module 403 over the pressurization module 402. The sanitization module 403 may include a UV-C source 406 and one or more filters 408 to sanitize the air before it is pressurized and pushed down toward the table 410. Thus, the configuration 400B may receive air from the external environment, sanitize, and pressurize to create the safe zone and the air shield.

In some embodiments, the alignment of the airflow may be directed in any desired direction in the XYZ axis. Incoming air may pass through a number of activated charcoal or HEPA filters. A plenum chamber may also house a number of UV-C sources (e.g., UV-C source 406) which shine on the inner surfaces of the HEPA filters to maintain their germ-free condition. The inlet air may be guided through a labyrinthine path to prevent the UV-C light from escaping to the outside as UV-C exposure is damaging to the eyes and skin. As HEPA filters may need to be regularly maintained/replaced to ensure proper operation, they may be easily accessible. An interlock may prevent the UV-C sources from operating when the plenum or any upper area is being serviced.

The fan 404 of the pressurization module 402 may be exclusively supplied with the pre-sanitized air (either from a sanitized air source or from the sanitization module). No exterior air may be allowed to be recirculated without passing through the filters. Exclusion or ingestion of ambient air is also prevented by the design of the containment enclosure chamber walls 414. The fan assembly may be capable of being adjusted either hard wired or wirelessly for airflow to allow for variances in users/guest count. Depending on size, the lower portion of the fan assembly zone may contain a variety of airflow straighteners, decorative covers or finger guards to prevent unwanted access to an area with moving parts. The fan assembly area may also contain a number of overhead and aimed lights for ambience. This area may also house some of the UV-C lighting used for cleanup following a dinner/meeting.

In some example embodiments, a roll-up cover 405 for UV-C cleaning may be implemented. Alternate methods may also be used. The working plenum area may include an aerodynamically shaped chamber wall 414 and the eating (or meeting) table 410. The chamber wall 414 may include a number of user openings 412 cut into it, with the dimensions determined by the size of the assembly as well as aerodynamic flow evaluation. Splitters 415 may be added for additional flow acceleration and extension of the exclusion boundary layer around the users. In other examples, multiple systems may be joined together to create a larger group device.

Diagrams 400C and 400D in FIG. 4B show the configurations of FIG. 4A with chairs 416 to allow users to sit at the table for eating or a meeting and be protected through the safe zone and air shields.

Sanitized air may be supplied to the system under high volume, with low velocity. The sanitized air may escape the containment enclosure through the openings 412 in the chamber wall 414. The users sit in the positive pressure area and are continuously enveloped in a column of sanitized air. Users may come into the restaurant wearing face coverings that may then be removed once they are in the positive flow zone. When the users leave the positive flow zone, face coverings may be put on again. When the meal is finished, the table may be bussed clean, the assembly may be covered to prevent light escape and internal UV-C lamps may be turned on for a preset time, completely cleaning all exposed surfaces. Once the lamps are turned off, the coverings may be raised/removed, the table may be set up again, and another set of users can be brought in for their meal.

As shown in diagram 400E of FIG. 4C, un-sanitized air 422 from the environment may be taken in through inlets at the top portion of the system and sanitized in a sanitization module 405 as described above. The sanitized air is then pushed down into the containment enclosure by the pressurization module (e.g., fan assembly 404). Two flows are generated: a low velocity, high pressure zone 426 in a central column; and a high velocity, low pressure zone 424 along the inner wall of the chamber (containment enclosure). The first flow prevents exchange of fluids and particulates between users sitting at openings, whereas the second flow creates an air shield for the users by escaping out of the openings with high velocity, low pressure. The escaped airflow has a downward angle allowing for attenuation of the effects within a short distance outside of the enclosure (and thereby allowing multiple installations in a particular area)

An example implementation environment for a system according to embodiments is a commercial restaurant setting. Thus, the system may additionally allow for a complete sanitization of all surfaces, rapid clean up and turn over for the next set of users. Multiple installations may take place in a normal restaurant (or similar) setting, for example, with 72-inch peripheries so as to maximize density without interfering or degrading adjoining devices. The system may also allow for the enjoyment of food, handling of documents, etc. without disturbing the items on a table through high speed air.

Diagrams 400F and 400G in FIG. 4D illustrate various fluid dynamic flow simulations for an example multi-user air safety system in a table configuration.

The computational fluid dynamics (CFD) simulations in following figures exhibit typical airflow through a multi-user air safety system according to embodiments. A table under a typical system with two outer wall openings is used for the simulation. The airflow is simulated at normal flow ratings and emanates from the fan assembly zone. For the simulation in diagram 400F, the flow volume is 400 CFM, which is adjustable as the layout dictates.

Diagram 400F includes a CFD pressure plot indicating areas of high and low pressure. The highest pressure 434 (and lowest airflow rate) is directly in the center of the table. The air intake 432 into the fan assembly zone (very low pressure) is indicated at the top of the system. The airflows away from the center in a radial pattern. This positive pressure away from the center prevents the passage of airborne fluids and particulates across the table. The pressure difference quickly decreases away from the table, reducing the impact on surrounding system installations. The outer wall constrains the pressure from doing a free escape into the room.

Diagram 400G is a CFD velocity plot indicating relative air speed and flow direction. The areas 444 indicate a flow speed of around 0.5 ft/sec (6 inches per second). A positive pressure and flow rate are maintained without washing out the area with high velocity air. The direction of flow is towards the floor, somewhat dissipated by a user seated in the air wash. The user is exposed to an airflow of approximately 0.75 ft/Sec (9″), or about ½ mile per hour as indicated by the areas 446. This flow rate does not generate excessive acoustic noise through turbulence. The airflow washes past the table edges through the gap between the table and the chamber wall. This allows the entire interior to bleed off pressure, maintaining a positive, balanced flow. The dark areas 442 demonstrate how well the flow dissipation is controlled in the vertical planes by showing the very low flow rate areas between the cutouts.

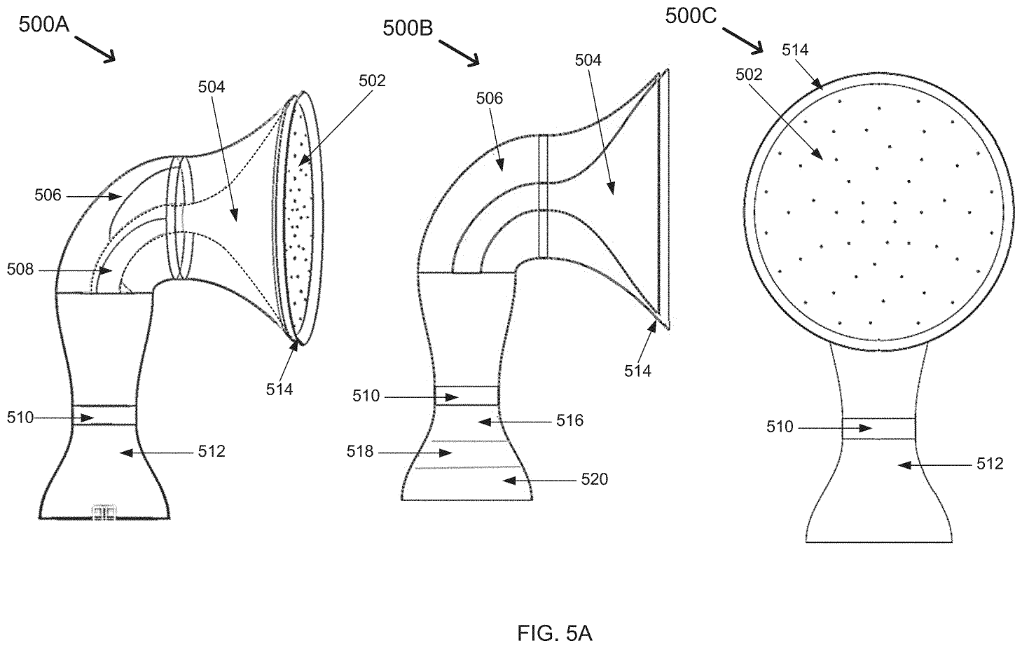

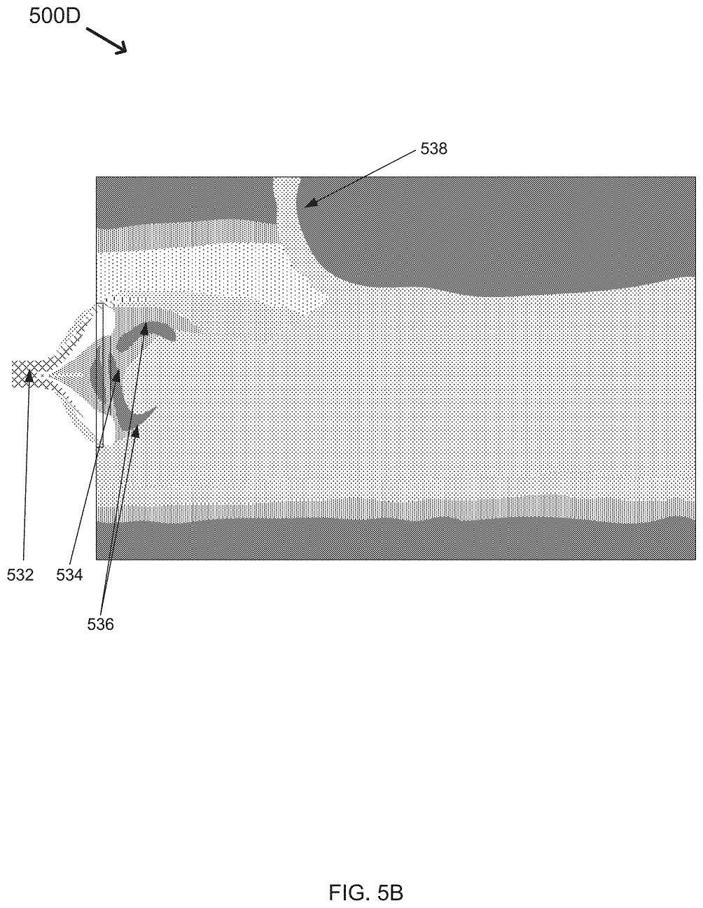

FIGS. 5A and 5B illustrate various views and fluid dynamic flow simulations for an example modular air safety system, arranged in accordance with at least some embodiments described herein.

A modular air safety system may provide clean air and prevent external contaminations for one or more people in a smaller or mobile environment. An example system, shown in a perspective view in diagram 500A of FIG. 5A may include a support stand 512 that contains pressurization module 510 (e.g., fan) and an airflow nozzle. The airflow nozzle may include a central flow portion with a shaped wall that defines an intake opening 508 and couples to an emitter opening 504 defined by the shaped wall. The emitter opening 504 may be covered by an emitter face 502, which includes a predefined number and size holes for the air to flow out. The coupling may be angled and have an increasing flow area. The airflow nozzle may also have a boundary flow portion surrounding the central flow portion and with another shaped wall that defines another intake opening 506 and couples to another emitter opening 514 defined by the other shaped wall. The second coupling may also be angled and have a decreasing flow area. The central flow portion may reduce a speed of received airflow and provide sanitized air toward a region of interest (e.g., user's face and/or upper body), and the boundary flow portion may increase a speed of received airflow and provide a conically shaped sanitized air shield around the region of interest (e.g., around the user's head or upper body).

The modular air safety system embodiments arrange high and low velocity outlets in a nozzle to accomplish the positive pressure and boundary flow, rather than the physical walls of a surround. Operationally similar to the multi-user air safety systems described herein, the modular system may essentially provide a short-range version of the multi-user system as air tends to diffuse rapidly. The support stand of the configuration shown in diagram 500A may include an air intake to receive pre-sanitized air from a sanitized air source and provide to the pressurization module 510. In other embodiments, atmospheric pressure air may be drawn from the environment into the modular air safety system through an inlet area 520 of the support stand. The received air may be passed through a UV-C light in area 518 that disinfects the filter 516 and the inlet area 520 as shown in diagram 500B of FIG. 5A. The pressurization module 510 pulls the sanitized air through the filter 516 and pressurizes it for both the central flow portion and the boundary flow portion. The airflow in the boundary flow portion is low velocity until it enters the compression area (emitter cone 514) where it is compressed by space reduction and then exits the airflow nozzle around the periphery of the emitter face 502. The central flow portion starts out with high speed airflow, which is then reduced by expansion when the air enters the emitter cone 504. The high volume, low velocity air for the central bubble is emitted through the emitter face 502. The support stand 512 may contain a joint to allow for rotational movement along one or more axes.

In some examples, a HEPA filter may capture any particulates bigger than 0.3 microns. The UV-C treatment may be provided by a UV lamp or LED and include failsafe mechanisms to prevent accidental exposure or a user's eyes and skin to UV-C light. To allow for comfortable communications within the safe zone, pressurization module (e.g., fan assembly) may be designed with a noise level below 50 dB. With a small footprint, the modular system may be used in environments such as offices, public encounter locations (e.g., banks, consumer stores, etc.), homes, hospitals, schools, vehicles (cars, buses, planes, ships, trains, etc.), and similar ones. While 50 dB is used as example noise limitation for an example system, embodiments are not limited to noise levels below 50 dB. Depending on implementation, noise mitigation measures may reduce to noise to suitable levels.

A shape and size of the modular air safety system may be selected and scaled depending on intended use and environment. For example, the substantially round airflow nozzle design shown in FIG. 5A may provide a safe zone for a single person, whereas a ‘flattened’ airflow nozzle design (substantially elliptical cross-section) may provide a wider safe zone (e.g. for two or more people). The joint between the support stand and the airflow nozzle (and/or parts of the support stand and the airflow nozzle) may be movable and/or flexible to allow adjustment of direction for the safe zone. Control elements for operation of the modular air safety system may be placed on the apparatus as desired or wired for remote operation. A power cord may be provided that is compatible with the voltage type and level used for the apparatus. The apparatus may be scaled for particular requirements, and may also be used either scaled, or in an array in a multi-user environment.

Diagram 500D in FIG. 5B illustrates fluid dynamic flow simulations for an example modular air safety system. The simulation in diagram 500D shows the protection of the central air “bubble” or safe zone 534 by the higher speed external flow field 536. The high pressure air being provided into the nozzle is indicated by reference numeral 532. Example flow rate is 120 CFM. In this case, a flow injection 538 simulating a human cough from the environment has been introduced to show how the boundary exclusion zone keeps the foreign material away from the sanitized air in the middle of the safe zone 534.

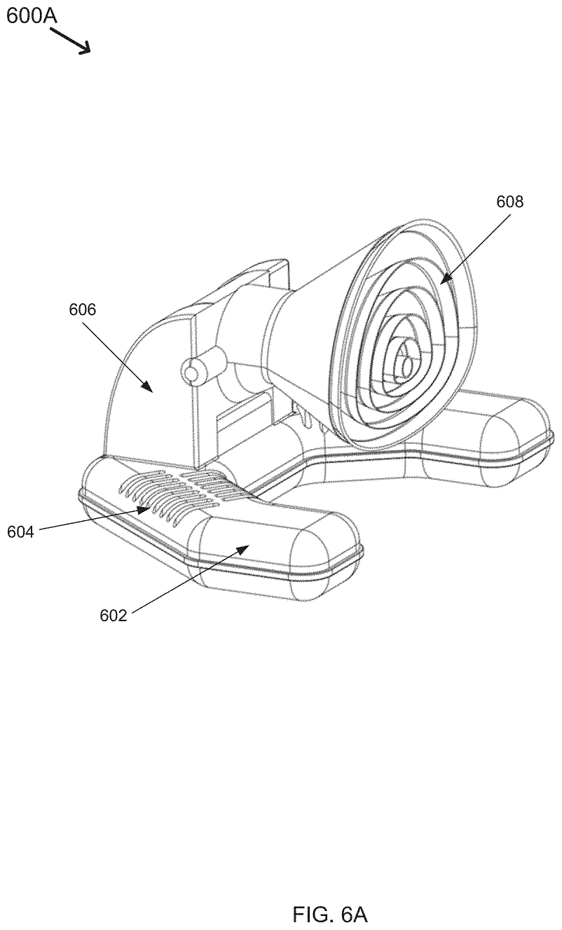

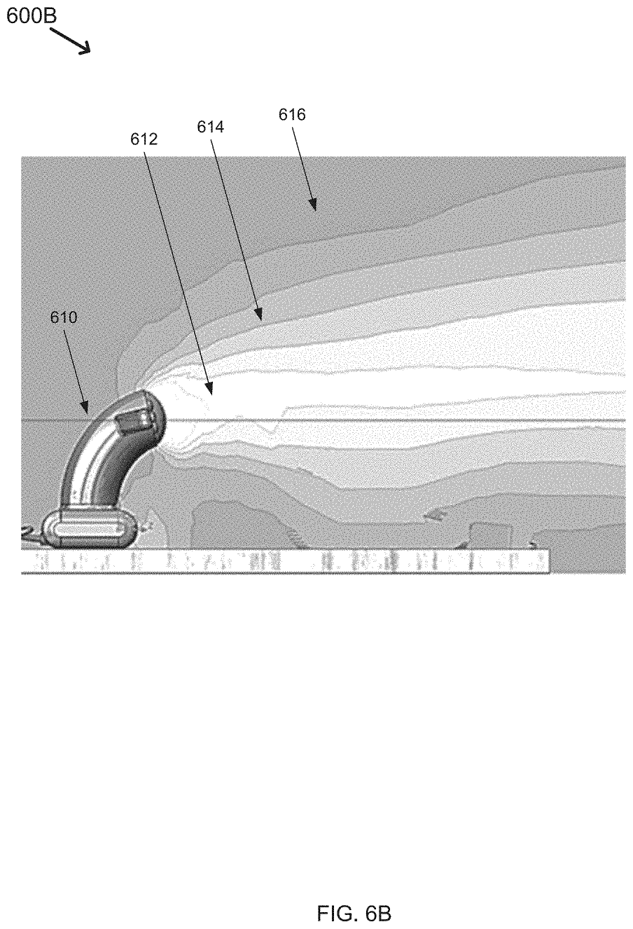

FIG. 6A illustrates a perspective view of another example modular air safety system, arranged in accordance with at least some embodiments described herein.

The example modular air safety system shown in diagram 600A includes an emitter face 608 shaped to generate a wider safe zone (as opposed to a conical) with multiple emitters arranged concentrically and the high velocity air shield flow being provided through the outermost emitter. The support stand includes to portions, a horizontal portion 602 comprising two legs for stability and a vertical portion 606 to support the airflow nozzle. Air intake openings 604 may be placed along the legs of the horizontal portion 602. The sanitization module and pressurization module, along with control elements, may be placed on either or both of the vertical portion 606 and the horizontal portion 602. The control elements may include turn-on/turn-off switches for different modules, adjustment for airflow, and an indicator showing airflow strength. The emitters may be arranged in any given number and shape to achieve an optimum safe zone. Depending on the desired safe zone, higher velocity air may be directed to some of the emitters (e.g., side emitters), whereas lower velocity air may be directed to the central emitters. The example modular air safety systems may be used in desktop implementations. They may also be attached to the back of a seat in a vehicle for protection of a passenger in the back seat, hung from a ceiling, or even configured as a wearable item.

FIG. 6B illustrates a fluid dynamic flow simulation for the example modular air safety system of FIG. 6A, arranged in accordance with at least some embodiments described herein.

Diagram 600B shows the air velocity distribution in the safe zone with darker shades showing lower speeds and lighter shades showing higher air speeds. As the plot shows, the modular air safety system 610 creates a safe zone of sanitized air in the central region 612 containing the user's head and/or upper body with the high velocity air shield 614 providing protection against any injection of fluids or particulates from the environment 616.

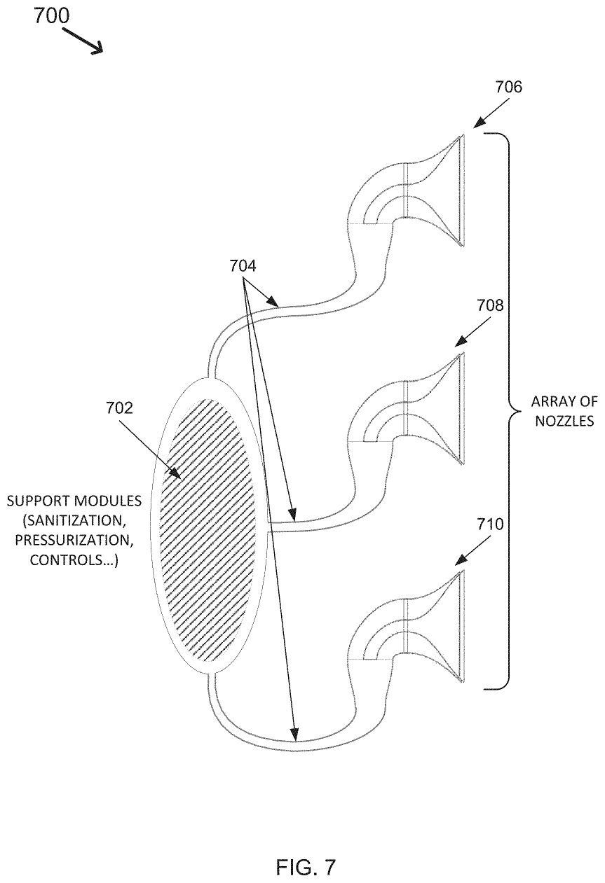

FIG. 7 illustrates an array configuration of the other example modular air safety system, arranged in accordance with at least some embodiments described herein.

In addition to adjustment of shape of the safe zone through the configuration of the airflow nozzle of a modular airflow system, the system may also be implemented with multiple airflow nozzles, for example arranged as an array, as shown in diagram 700. A common support module 702 may contain power circuitry, sanitization module(s), pressurization module(s), and/or controls. Any number of airflow nozzles (706, 708, 710) may be mechanically coupled to the common support module 702 through hoses, pipes, or similar couplings 704 such that the airflow nozzles 706, 708, 710 can be arranged as desired in an environment for multiple users. For example, three airflow nozzles may be arranged in front of three tellers at a bank supported by the same support module. In another example, airflow nozzles may be arranged for individual passengers in a vehicle (e.g., car, train, plane, etc.) and coupled to a central support module providing protection to each passenger individually.

As mentioned herein, various components of the multi-user and modular air safety systems may be made from various synthetic materials such as PVC, polypropylene, ceramic, or metals such as aluminum, stainless steel, and other materials. Various surfaces may be treated or made from anti-microbial materials. Different color and/or surface texture schemes may be used.

According to some examples, an air safety system may include an air source configured to receive air and direct the air to the air safety system; a pressurization module configured to receive the air from the air source and direct the air at a predetermined pressure to a guidance structure; and the guidance structure comprising two or more channels. The guidance structure, through the two or more channels, may be configured to generate a first airflow in a central column region with a first pressure to create a safe zone with low velocity, sanitized air for one or more users within the central column region; and generate a second airflow to form an air shield around the central column region with a second pressure higher than the first pressure to prevent entry of fluids and particulates from an external environment into the safe zone. The air safety system may include an air source configured to receive pre-sanitized air and direct the pre-sanitized air to the air safety system. Alternatively, the air source may be an air intake module configured to receive air from the external environment, where the air safety system may further include a sanitization module comprising one or more of a filter module to filter the received air and an ultra-violet (UV) module to apply UV light to the received air. The sanitization module may sanitize the received air as the air passes from the air source to the pressurization module.

According to other examples, the first airflow in the central column region with the first pressure may be configured to prevent interchange of fluids and particulates between the two or more users within the safe zone. The system may also include an air source configured to receive pre-sanitized air and direct the pre-sanitized air to the air safety system. The air source may be an air intake module configured to receive air from the external environment, and the air safety system may further include a sanitization module comprising one or more of a filter module to filter the received air and an ultra-violet (UV) module to apply UV light to the received air. The sanitization module may be configured to sanitize the received air as the air passes from the air source to the pressurization module. The UV module may include a lamp or a light emitting diode (LED) to provide UV-C light. The pressurization module may include one or more fans or an air multiplier. The pressurization module may include noise control measures to keep a noise level below about 50 dB. The pressurization module may be configured to adjust one or more of the first pressure and the second pressure based on one or more of a number of present users, a usage environment, or a user preference. The air safety system may be configured to generate the central column region and the air shield vertically, horizontally, or at an angle.

According to further examples, the air safety system may further include a containment structure comprising a hollow chamber and two or more user openings, where the containment structure is configured to be placed on a table, and the central column region is formed inside the hollow chamber and the air shield is formed outside the two or more user openings. The hollow chamber may be shaped as a cylinder, a paraboloid, or a cone. Walls of the hollow chamber may be transparent, semi-transparent, or opaque. The two or more user openings may have a cross section shaped substantially as a circle, an ellipse, a triangle, a parabola, a rectangle, or a combination thereof. A shape and a size of the hollow chamber and the two or more user openings may be selected or adjusted based on one or more of a size of a table within the hollow chamber, a number of openings, or a capacity of the pressurization module. The air safety system may further include a UV module configured to treat an interior of the hollow chamber between uses; and a cover configured to be lowered around an exterior of the hollow chamber when the UV module is in use.

According to other examples, a modular air safety system may include a support module with an air intake opening configured to receive air; a pressurization module to direct the air at a predetermined positive pressure; and one or more airflow nozzles coupled to the support module through a joint. The one or more airflow nozzles may include a central flow portion having a shaped wall that defines an intake opening and couples to an emitter opening defined by the shaped wall, where the coupling is angled and has an increasing flow area; and a boundary flow portion surrounding the central flow portion and having another shaped wall that defines another intake opening and couples to another emitter opening defined by the other shaped wall, where the coupling is angled and has a decreasing flow area. The central flow portion may be configured to reduce a speed of received airflow and provide sanitized air toward a region of interest, and the boundary flow portion may be configured to increase a speed of received airflow and provide an air shield around the region of interest to prevent entry of fluids and particulates from an external environment.

According to some examples, the air intake opening may be configured to receive pre-sanitized air and direct the pre-sanitized air to the pressurization module. The air intake module may be configured to receive the air from the external environment, and the support module may include a sanitization module comprising one or more of a filter module to filter the received air and an ultra-violet (UV) module to apply UV light to the received air. The sanitization module may be configured to sanitize the received air as the air passes from the air intake opening to the pressurization module. The UV module may include a lamp or a light emitting diode (LED) to provide UV-C light. The pressurization module may include one or more fans or an air multiplier. The pressurization module may include noise control measures to keep a noise level below about 50 dB. The pressurization module may be configured to adjust one or more of the predetermined positive pressure based on one or more of a number of present users, a usage environment, or a user preference.

According to other examples, the one or more airflow nozzles may be configured to generate the air shield around a vertical or horizontal region of interest. The joint may be configured to provide a directional adjustment to the airflow nozzle along one or more axes. A shape of the one or more airflow nozzles may be substantially one of cylindrical, conical, parabolical, or rectangular. A size of the one or more airflow nozzles may be selectable based on one or more of a distance to the user, a number of users, a user preference, a shape of a safe zone to be created, a size of the safe cone, or a usage environment. One or more surfaces of the airflow nozzles and the support module may be treated with or made from anti-microbial material. The support module may house one or more operational control elements.

According to further examples, a method to create a safe zone with sanitized air may include receiving pre-sanitized air from a pre-sanitized air source; pressurizing the received pre-sanitized air at a predetermined pressure to a guidance structure; generating a first airflow in a central column region with a first pressure to create a safe zone with low velocity, sanitized air for one or more users within the central column region by guiding the pressurized air through one or more channels of the guidance structure; and generating a second airflow to form an air shield around the central column region with a second pressure higher than the first pressure to prevent entry of fluids and particulates from an external environment into the safe zone by guiding the pressurized air through one or more other channels of the guidance structure.

According to yet other examples, the method may also include selecting the first pressure to prevent interchange of fluids and particulates between the two or more users within the safe zone. Pressurizing the received pre-sanitized air at the predetermined pressure to the guidance structure may include pressurizing the pre-sanitized air through one or more fans or an air multiplier. The method may further include employing noise control measures to keep a noise level below about 50 dB. Pressurizing the received pre-sanitized air at the predetermined pressure to the guidance structure may include adjusting one or more of the first pressure and the second pressure based on one or more of a number of present users, a usage environment, or a user preference. Generating the second airflow to form the air shield may include generating the air shield vertically, horizontally, or at an angle.

According to some examples, a method to create a safe zone with sanitized air may include receiving air from an external environment; sanitizing the received air through a sanitization module comprising one or more of a filter module to filter the received air and an ultra-violet (UV) module to apply UV light to the received air; pressurizing the sanitized air at a predetermined pressure to a guidance structure; generating a first airflow in a central column region with a first pressure to create a safe zone with low velocity, sanitized air for one or more users within the central column region by guiding the pressurized air through one or more channels of the guidance structure; and generating a second airflow to form an air shield around the central column region with a second pressure higher than the first pressure to prevent entry of fluids and particulates from an external environment into the safe zone by guiding the pressurized air through one or more other channels of the guidance structure.

According to yet other examples, the UV module may include a lamp or a light emitting diode (LED) to provide UV-C light. The method may also include selecting the first pressure to prevent interchange of fluids and particulates between the two or more users within the safe zone. Pressurizing the sanitized air at the predetermined pressure to the guidance structure may include pressurizing the sanitized air through one or more fans or an air multiplier. The method may further include employing noise control measures to keep a noise level below about 50 dB. Pressurizing the sanitized air at the predetermined pressure to the guidance structure may include adjusting one or more of the first pressure and the second pressure based on one or more of a number of present users, a usage environment, or a user preference. Generating the second airflow to form the air shield may include generating the air shield vertically, horizontally, or at an angle.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only and is not intended to be limiting.

The herein described subject matter sometimes illustrates different components contained within, or connected with, different other components. Such depicted architectures are merely examples, and in fact, many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

1. An air safety system comprising:
an air source configured to receive air and direct the air to the air safety system;
a pressurization module configured to receive the air from the air source and direct the air at a predetermined pressure to a guidance structure; and
the guidance structure comprising two or more channels, the guidance structure, through the two or more channels, configured to:
generate a first airflow in a central column region with a first pressure to create a safe zone with low velocity, sanitized air for one or more users within the central column region; and
generate a second airflow to form an air shield around the central column region with a second pressure higher than the first pressure to prevent entry of fluids and particulates from an external environment into the safe zone.

2. (canceled)

3. The air safety system of claim 1, further comprising:
an air source configured to receive pre-sanitized air and direct the pre-sanitized air to the air safety system.

4. The air safety system of claim 1, wherein the air source is an air intake module configured to receive air from the external environment, and the air safety system further comprises:
a sanitization module comprising one or more of a filter module to filter the received air and an ultra-violet (UV) module to apply UV light to the received air, the sanitization module configured to:
sanitize the received air as the air passes from the air source to the pressurization module.

5. (canceled)

6. The air safety system of claim 1, wherein the pressurization module includes one or more fans or an air multiplier.

7. (canceled)

8. The air safety system of claim 1, wherein the pressurization module is configured to adjust one or more of the first pressure and the second pressure based on one or more of a number of present users, a usage environment, or a user preference.

9.-10. (canceled)

11. The air safety system of claim 1, further comprising:
a containment structure comprising a hollow chamber and two or more user openings, wherein
the containment structure is configured to be placed on a table, and
the central column region is formed inside the hollow chamber and the air shield is formed outside the two or more user openings.

12. The air safety system of claim 11, wherein
the hollow chamber is shaped as a cylinder, a paraboloid, or a cone,
walls of the hollow chamber are transparent, semi-transparent, or opaque, and
the two or more user openings have a cross section shaped substantially as a circle, an ellipse, a triangle, a parabola, a rectangle, or a combination thereof.

13.-16. (canceled)

17. A modular air safety system comprising:
a support module comprising:
an air intake opening configured to receive air;
a pressurization module to direct the air at a predetermined positive pressure; and
one or more airflow nozzles coupled to the support module through a joint, the one or more airflow nozzles comprising:
a central flow portion having a shaped wall that defines an intake opening and couples to an emitter opening defined by the shaped wall, wherein the coupling is angled and has an increasing flow area; and
a boundary flow portion surrounding the central flow portion and having another shaped wall that defines another intake opening and couples to another emitter opening defined by the other shaped wall, wherein the coupling is angled and has a decreasing flow area, wherein
the central flow portion is configured to reduce a speed of received airflow and provide sanitized air toward a region of interest, and
the boundary flow portion is configured to increase a speed of received airflow and provide an air shield around the region of interest to prevent entry of fluids and particulates from an external environment.

18. The modular air safety system of claim 17, wherein the air intake opening is configured to receive pre-sanitized air and direct the pre-sanitized air to the pressurization module.

19. The modular air safety system of claim 17, wherein the air intake module is configured to receive the air from the external environment, and the support module further comprises:
a sanitization module comprising one or more of a filter module to filter the received air and an ultra-violet (UV) module to apply UV light to the received air, the sanitization module configured to:
sanitize the received air as the air passes from the air intake opening to the pressurization module.

20. (canceled)

21. The modular air safety system of claim 17, wherein the pressurization module includes one or more fans or an air multiplier.

22. (canceled)

23. The modular air safety system of claim 17, wherein the pressurization module is configured to adjust one or more of the predetermined positive pressure based on one or more of a number of present users, a usage environment, or a user preference.

24. The modular air safety system of claim 17, wherein the one or more airflow nozzles are configured to generate the air shield around a vertical or horizontal region of interest.

25. The modular air safety system of claim 17, wherein the joint is configured to provide a directional adjustment to the airflow nozzle along one or more axes.

26. The modular air safety system of claim 17, wherein a shape of the one or more airflow nozzles is substantially one of cylindrical, conical, parabolical, or rectangular.

27.-28. (canceled)

29. The modular air safety system of claim 17, wherein the support module houses one or more operational control elements.

30. A method to create a safe zone with sanitized air, the method comprising:
receiving pre-sanitized air from a pre-sanitized air source;
pressurizing the received pre-sanitized air at a predetermined pressure to a guidance structure;
generating a first airflow in a central column region with a first pressure to create a safe zone with low velocity, sanitized air for one or more users within the central column region by guiding the pressurized air through one or more channels of the guidance structure; and
generating a second airflow to form an air shield around the central column region with a second pressure higher than the first pressure to prevent entry of fluids and particulates from an external environment into the safe zone by guiding the pressurized air through one or more other channels of the guidance structure.

31. The method of claim 30, further comprising:
selecting the first pressure to prevent interchange of fluids and particulates between the two or more users within the safe zone.

32. The method of claim 30, wherein pressurizing the received pre-sanitized air at the predetermined pressure to the guidance structure comprises pressurizing the pre-sanitized air through one or more fans or an air multiplier.

33. (canceled)

34. (canceled)

35. The method of claim 30, wherein generating the second airflow to form the air shield comprises generating the air shield vertically, horizontally, or at an angle.

36.-42. (canceled)
